(19) United States
(12) Patent Application Publication
(10) Pub. No.: US 2007/0214012 A1
(43) Pub. Date: Sep. 13, 2007

(54) METHOD OF USING ONLINE COMMUNITIES FOR EFFECTIVE CLINICAL TRIAL RECRUITMENT

(76) Inventors: Brian LOEW, Arlington, VA (US); Amir Lewkowicz, Arlington, VA (US)

Correspondence Address: MCGUIREWOODS, LLP, 1750 TYSONS BLVD, SUITE 1800, MCLEAN, VA, 22102, US

(21) Appl. No.: 11/685,591

(22) Filed: Mar. 13, 2007

Related U.S. Application Data
(60) Provisional application No. 60/781,322, filed on Mar. 13, 2006.

(51) Int. Cl. G06Q 10/00; A61B 5/00

(52) U.S. Cl. 705/2; 600/300

(57) ABSTRACT

A method of recruiting clinical trial candidates using an online community is disclosed that includes creating an illness-specific online community. Membership accounts are created for members joining the online community. Personal and medical information is collected from the members and categorized and stored in a member database. Information on a clinical trial is collected from a client. The member database is searched to determine candidates for the clinical trial.

### CROSS REFERENCE TO PRIOR APPLICATION

This application claims priority to and the benefit of U.S. Provisional Patent Application No. 60/781,322 filed on Mar. 13, 2006, which is hereby incorporated by reference for all purposes as if fully set forth herein.

### BACKGROUND OF THE INVENTION

1. Field of the Invention

The invention is directed to a method of using online communities for effective clinical trial recruitment to both improve the lives of individuals and to advance medicine through effective clinical trials.

2. Related Art

In 2004, U.S. pharmaceutical companies invested $39 billion on research & development. Of that investment, $18 billion was spent on clinical development, 16% of which, or just under $3 billion, was spent on the recruitment of suitable patients for U.S. States, 80% of which are delayed significantly due to unfulfilled enrollment. Patient recruitment accounts for 30% of time spent on clinical trials. Due to the challenge of finding suitable participants, 94% of clinical trials miss their enrollment deadlines in the U.S. Today's methods of recruiting participants have changed little in the past 20 years, and only 4%-6% of eligible patients who suffer from severe and life-threatening illnesses take part in U.S. clinical trials.

Currently clinical trial recruitment candidates are found through conventional mass media advertising, database/data mining, online listings, or via contact with hospitals/medical centers/physicians. Even with all these recruitment methods, there are still significant challenges in recruiting suitable patients in a timely manner. Recruitment for Phase III trials is the most challenging, often requiring 18 months to complete. These trials, which are the largest, involve 2,000 to 4,000 patients, and occur after efficacy has already been demonstrated. Delays in Phase III recruitment are frustrating and expensive.

Accordingly, there is a need for an effective process to recruit participants for clinical trials from a large pool of potential candidates.

### SUMMARY OF THE INVENTION

The invention meets the foregoing need and provides an effective method of recruiting participants for clinical trials from a large pool of potential candidates, which results in significantly quicker drug trials and other advantages apparent from the discussion herein.

Accordingly, in one aspect of the invention, a method of recruiting clinical trial candidates using an online community includes the steps of creating an illness-specific online community; creating membership accounts for members joining the online community; receiving personal and medical information from the members; categorizing and storing the received personal and medical information to a member database; receiving information on a clinical trial from a client; and searching the member database to determine candidates for the clinical trial.

According to another aspect of the invention, a method of recruiting clinical trial participants online includes the steps of creating a website dedicated to at least one specific illness, wherein the website includes a plurality of web pages linked to each other, the web pages including a home page, membership application page and member profile page; displaying the membership application page containing an online membership application form when an online visitor indicates that he or she wants to be a member; creating a membership account for the member responsive to a visitor's input; collecting personal and medical information from the member; associating the member's personal and medical information to the member's membership account; categorizing and storing the member's personal and medical information to a member database; receiving clinical trial information from a client; comparing the clinical trial information to the member's personal and medical information to determine if the member is a candidate for the client's clinical trial; and contacting the member to offer an opportunity to participate in the client's clinical trial.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention. No attempt is made to show structural details of the invention in more detail than may be necessary for a fundamental understanding of the invention and the various ways in which it may be practiced. In the drawings:

FIG. 1 shows an exemplary flow chart for recruiting clinical trial candidates from an on-line community in response to a client's requirements according to the principles of the invention;

FIG. 2 shows an exemplary partial screen capture image of an exemplary home page of an on-line illness community constructed according to the principles of the invention;

FIG. 3 shows an exemplary first partial screen capture image of an exemplary member registration page (upper portion) of the on-line illness community of FIG. 2;

FIG. 4 shows an exemplary second partial screen capture image of the exemplary member registration page (lower portion) of FIG. 3;

FIG. 5A shows an exemplary third partial screen capture image of the exemplary member registration page of FIG. 3, in which an exemplary first pull-down menu feature is implemented to select a condition of interest;

FIG. 5B shows an exemplary fourth partial screen capture image of the exemplary member registration page of FIG. 3, in which an exemplary second pull-down menu feature is implemented to select a reason for the interest in the condition selected in FIG. 5A;

FIG. 5C shows an exemplary fifth partial screen capture image of the exemplary member registration page of FIG. 3, in which an exemplary third pull-down menu feature is implemented to select a treatment of interest;

FIG. 6 shows an exemplary partial screen capture image of an exemplary main member profile page of the on-line illness community of FIG. 3;

FIG. 7 shows an exemplary partial screen capture image of an exemplary member profile page for editing medical information linked from the exemplary main member profile page of FIG. 6;

FIG. 8A shows an exemplary first partial screen capture image of another exemplary member profile page for selecting sharing and security options linked from the exemplary main member profile page of FIG. 6; and

FIG. 8B shows an exemplary second partial screen capture image of the exemplary member profile page of FIG. 8A.

### DETAILED DESCRIPTION OF THE INVENTION

The embodiments of the invention and the various features and advantageous details thereof are explained more fully with reference to the non-limiting embodiments and examples that are described and/or illustrated in the accompanying drawings and detailed in the following description. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals represent similar parts throughout the several views of the drawings.

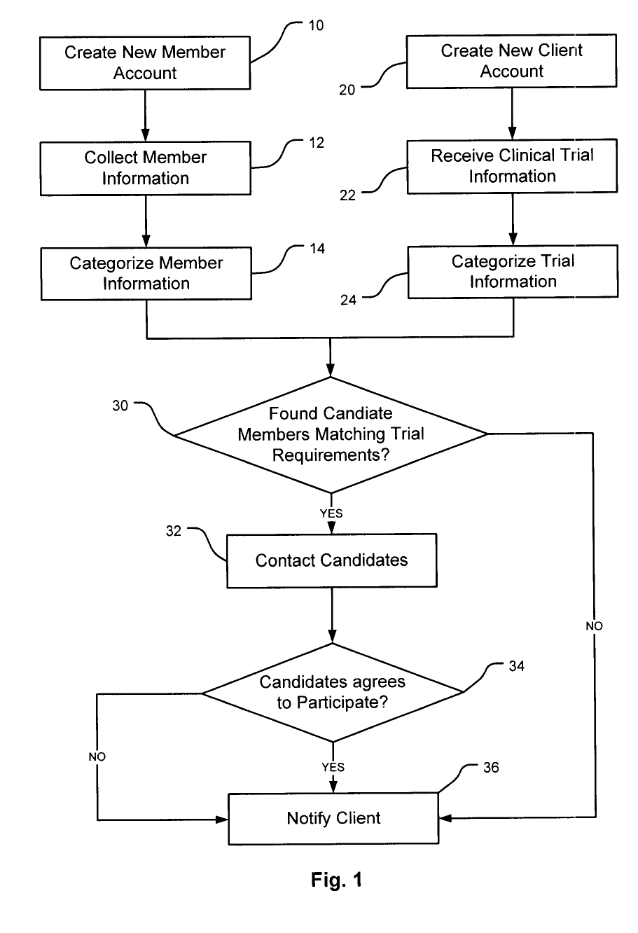

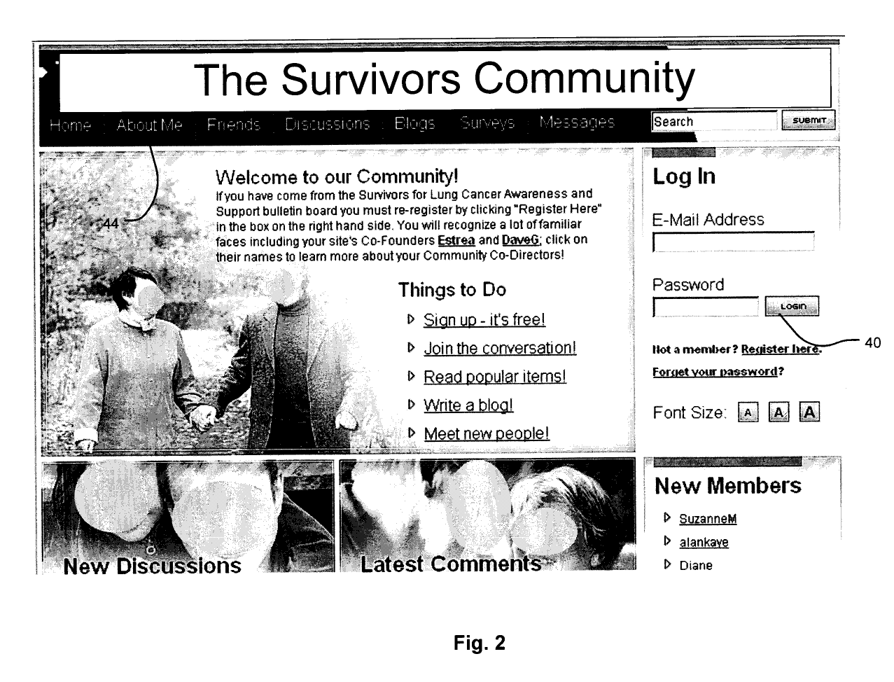

FIG. 1 shows a flow chart describing an overall process for recruiting participants for clinical trials using an on-line community. While the invention may be implemented in any large online portals such as Yahoo, Google, MSN, AOL, etc., a smaller and illness-specific online community may be more effective in finding and recruiting participants for clinical trails for specific illness types. For example, FIG. 2 shows a partial screen capture image of a home page of The Survivors Community, which focuses on issues related to lung cancer. The on-line community offers a virtual space for anyone to meet other cancer patients or cancer survivors, share personal experience and recent medical developments, develop friendships with other members, and help and support other cancer patients or patents' family members or friends. Note, the invention is directed to any type on-line community.

The website may be configured such that any visitor can browse to find general or specific information and read other members' blogs and/or discussions and so on without becoming a member or the access may be partially limited to members. Also, the website may be configured such that only registered members can create their own blogs, participate discussions and the like.

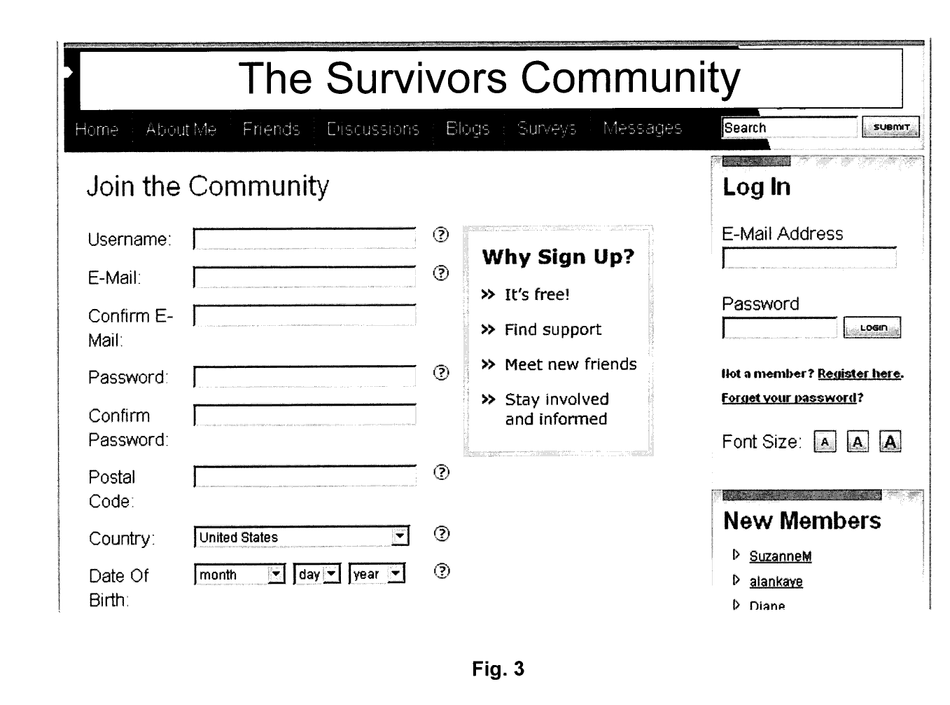

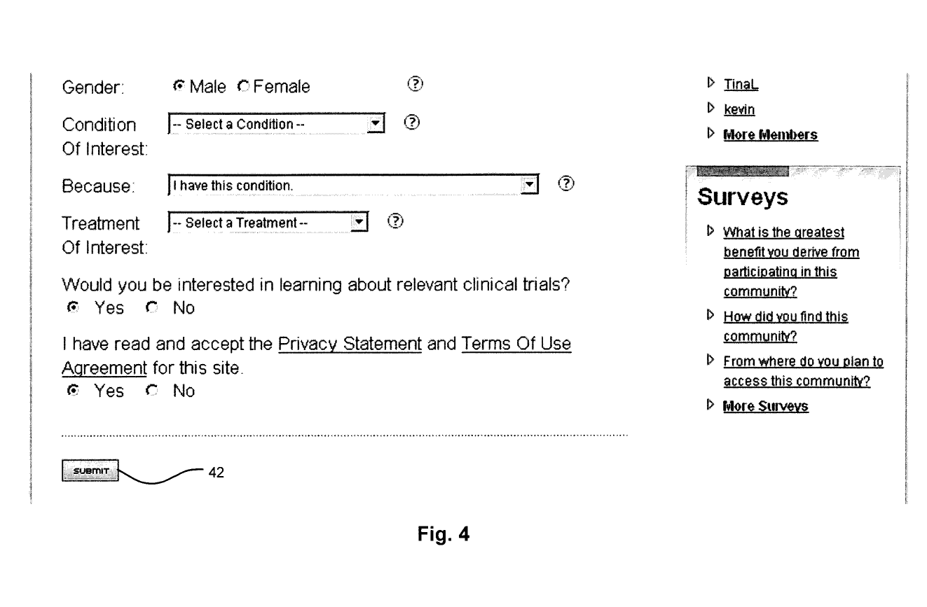

For an applicant who wants to become a registered member, a link 40 captioned “Register here” or the like may be provided on the home page shown in FIG. 2. By clicking the link 40, a member registration page including an on-line member registration form, such as one shown in FIGS. 3 and 4, is displayed on the screen. FIG. 3 shows the top portion of the member registration page where the applicant can submit basic personal information such as email address, postal code, country and date of birth, choose login information such as user name and password and so on. FIG. 4 shows the bottom portion of the member registration page where the applicant may submit more specific information, if he or she is inclined to do so, such as condition of interest and treatment of interest. Also, the member registration page may include a question regarding whether the applicant would be interested in learning about relevant clinical trials, as shown in FIG. 4. Of course the registration page may include more or less features and such is contemplated by the invention.

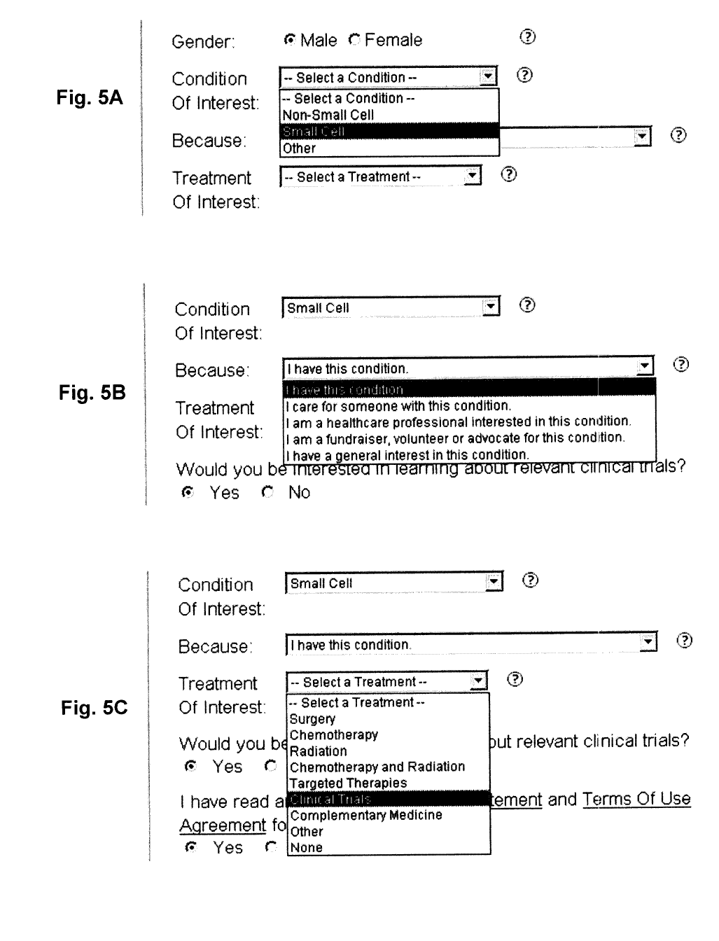

The information gathered from the registration form may be useful in determining whether he or she is a candidate for clinical trails for specific symptoms. For example, FIGS. 5A, 5B, 5C show partial screen shot images of the bottom portion of the member registration page shown in FIG. 4, where a pull-down menu feature is implanted for selecting categories matching the applicant's conditions and interests. Specifically, FIG. 5A shows three options available on the pull down menu for the “Condition Of Interest,” which are “Non-Small Cell,” “Small Cell” and “Other.” FIG. 5B shows four options in the pull down menu for the reason why the applicant is interested in the selected “Condition of Interest,” which are “I have this condition,” “I care for someone with this condition,” “I am a healthcare professional interested in this condition,” “I am a fundraiser, volunteer or advocate for this condition” and “I have a general interest in this condition.” If the applicant selects “Non-Small Cell” or “Small Cell” for the condition of interest in FIG. 5A and selects “I have this condition” as the reason for being interested in the condition in FIG. 5B, he or she may be a candidate for clinical trials. FIG. 5C shows nine options provided for the “Treatment of Interest,” which are “Surgery,” “Chemotherapy,” “Radiation,” “Chemotherapy and Radiation,” “Targeted Therapies,” “Clinical Trials,” “Complementary Medicine,” “Other” and “None.” If the applicant selects “Clinical Trial,” it may be a strong indication that he or she is willing to participate in clinical trials pertaining to his or her symptom.

Upon completing the member registration form and clicking a “submit” button 42 or the like shown in FIG. 4, the information provided by the applicant is transferred via Internet to and stored in a database managed by the on-line community management. If there are no issues with the information submitted by the applicant, a new membership account is created for the applicant, as indicated as a step 10 in FIG. 1, and the information provided by the applicant is associated with his or her membership account.

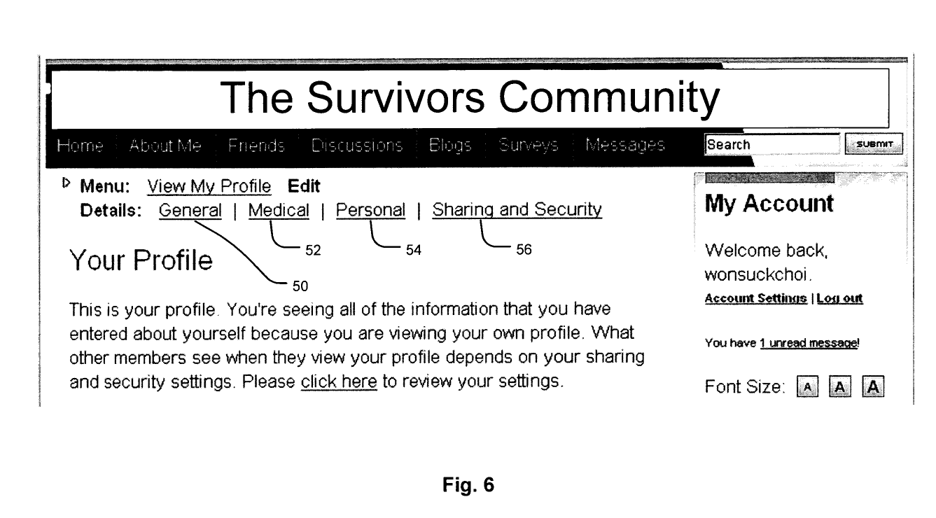

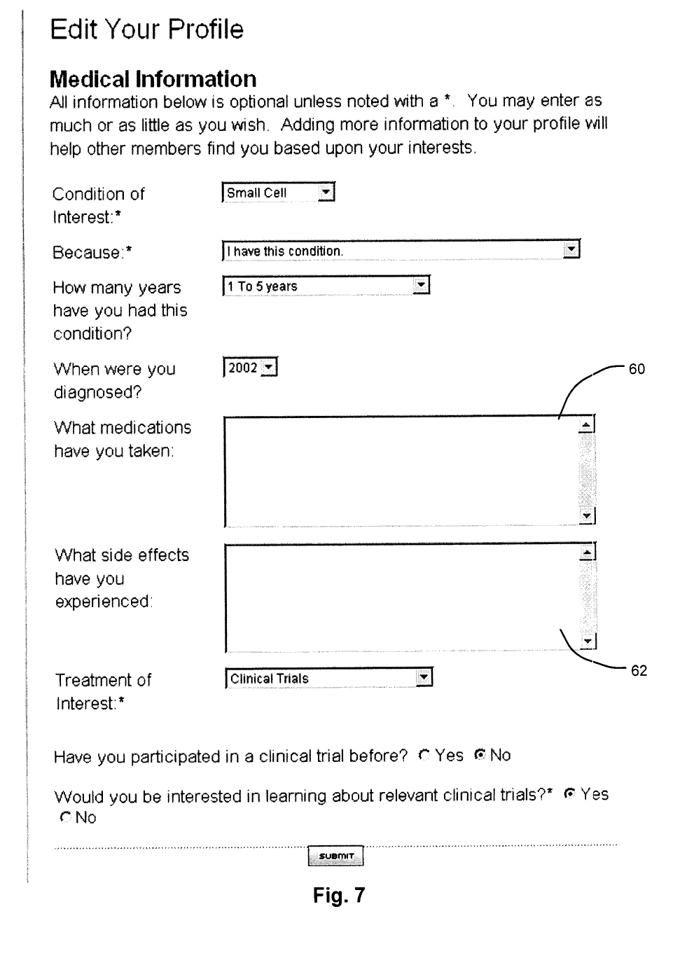

Once the new account is created, the member is provided with an option to submit more detailed personal and medical information, which may be necessary to determine whether he or she is a candidate for certain clinical trails, which is shown as the step 12 in FIG. 1. The member may be allowed to edit, delete and/or add the information he or she previously provided by logging in to the website. For example, the home page shown in FIG. 2 is provided a link 44 captioned “About me” or the like. By clicking the link 44, a member profile page, such as one shown in FIG. 6, may be displayed. The member profile page shown in FIG. 6 includes several links 50, 52, 54, 56 to more specific member profile pages. The links 50 and 54 captioned “General” and “Personal” are linked to profile pages (not shown) where the member may review and edit the member's general and personal information, such as name, address, phone number, hobby, etc. The link 52 captioned “Medical” is linked to the member's medical profile page such as one shown in FIG. 7. The member's medical profile page may display the condition and interests previously selected by the member when he or she applied for the membership as described above in conjunction with FIGS. 4, 5A, 5B, 5C, such as “Condition of Interest,” “Because” and Treatment of Interest.” Also, the medical profile page allows the member to submit more information such as “How many years have you had this condition?” and “When were you diagnosed?” These categories may also be implanted with a full-down menu for categorizing the member's medical information. The medical profile page may also have questions such as “What medications have you taken?” and “What side effects have you experienced?” The member can answer these questions by typing in the text boxes 60, 62, respectively, which may be considered for further analysis once the member is determined to be a candidate for certain clinical trials. The medical profile page may further include questions such as “Have you participated in a clinical trial before?” and “Would you be interested in learning about relevant clinical trials?” The member may select the answers to these questions by selecting one of “Yes” or “No” provided next to the questions.

To protect the member's privacy, the website may provide the member with an option on how he or she wants his or her personal and medical information shared with other members or third parties including pharmaceutical companies who are looking for clinical trial candidates. If the member chooses not to share his or her information, no member information is displayed on the website or provided to the third parties. Also, the member may choose to partially share his or her personal or medical information. For example, the member may choose to share his name and email address but to hide his medical information such as the information submitted in the member medical profile shown in FIG. 7.

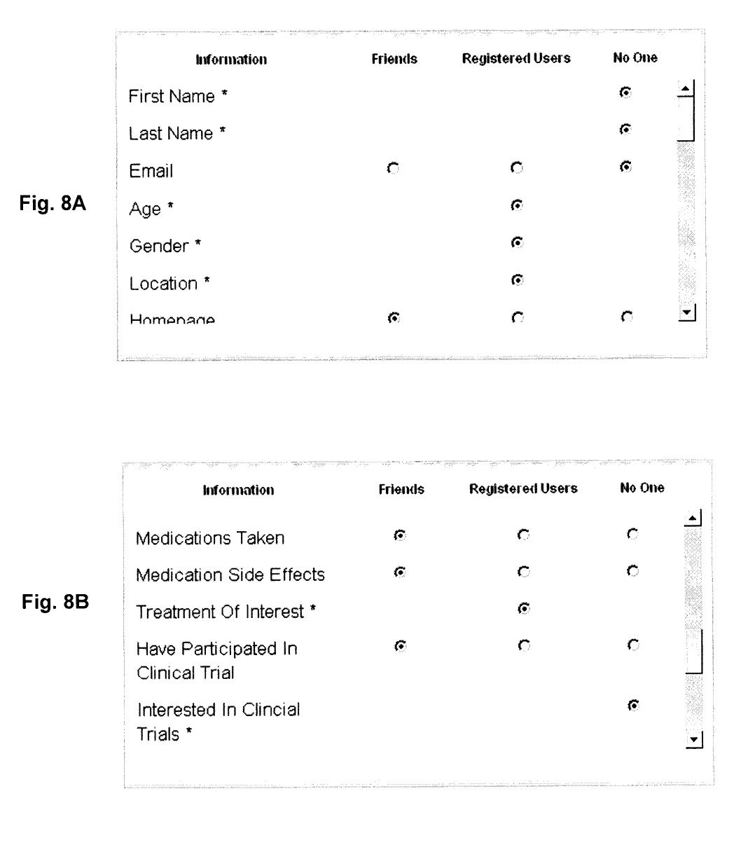

FIGS. 8A and 8B show partial screen capture images of a sharing and security setup page where the member can choose the personal and medical information he or she wants to share. The sharing and security setup page may be linked from the link 56 of the main member profile page shown in FIG. 6. As shown therein, the member may selectively allow others to access his or her personal and medical information. For example, the member may elect not to disclose his or her email address to anyone by selecting “No One” while allowing only his or her friends to access information related to the member's “home page,” “medication taken,” “medication side effects” and “Have Participated In Clinical Trial” by selecting “Friends.” Particularly, FIG. 8B shows that the member selected “No One” regarding whether he or she is “Interested in Clinical Trials.” In this case, the information on whether the member is interested in clinical trial is strictly restricted from access to protect the member's privacy.

Steps 10, 12 and 14 in FIG. 1 are repeated every time a new visitor applies for the membership, and information collected from the members may be categorized and saved to the database, as shown in the step 14 of FIG. 1.

Concurrently with or independently from building the member database by collecting and categorizing information from the members as shown in the steps 10, 12 and 14 of FIG. 1, the online community may offer a clinical trial recruiting service customized for a client. The client may be a pharmaceutical company, clinical trial recruiting agency, hospital, University, government, etc. For example, the client may be a pharmaceutical company that is in the process of developing a new drug for lung cancer. The client may contact the online community management at any stage of the new drug development. For example, the client may contact the online community management prior to conducting research and development for a new drug or after the new drug is developed. To start a process of finding clinical trial candidates, a new client account may be opened, which is shown as step 20 in FIG. 1. Upon opening a new account, information on the client's clinical trail is collected, which is shown as a step 22. The clinical trial information collected from the client is categorized and stored to a client database, which is shown as a step 24.

Upon completing categorizing the clinical trial information from the client, a process for finding members who match the requirements for the client's clinical trial, which is shown as a step 30 in FIG. 1. In doing so, the client database may be interrelated with the member database such that a list of the members matching the client's trial requirements is automatically generated. For example, if the client is interested in finding a member who actually has a small cell condition and is interested in participating in clinical trials, a simple member database search would result in a full list of the matching members in the member database. The client may be regularly notified on the progress of the process of finding candidates. For example, the online community may provide a daily, weekly, monthly, quarterly and/or yearly report to the client. If no matching candidates are found from the database for a certain period time, the client may be notified accordingly such that the client may decide to pursue additional recruiting options.

One advantage of the invention is that the categorized member information has been accumulated in the member database from the beginning of the online community, and hence the online community may instantly provide the list of candidates that matches the client's trial requirements. Also, since the member information is categorized, the online community may provide a customized search report to the client, which may be sorted by the members' personal and medical information. For example, the online community may provide a list of trial candidates based on their sex, age, geographical location, condition, treatment of interested, etc. It should be noted that the members who have chosen not to share their medical information may not be included in the list of candidates provided to the client.

Once the trial candidates are identified, a process of contacting each of the candidates is initiated in order to offer the candidates an opportunity to participate the client's clinical trial, which is shown as a step 32 in FIG. 1. Although the contact process may be performed by the online community as shown in FIG. 1, the process may be performed by the client or the client's agent or contactors. In fact, the client or their agent may be more effective in preparing and presenting information about their clinical trial because usually the client has more detailed medical information on their trials.

The candidate contact process may be performed by mail, email, phone call, invitation to an event, etc. The candidates who are interested in participating in the trial may contact the online community or the client by mail, email, phone call, etc. The contact process may be continued only for a limited time period, and the online community may provide the client with a periodic progress report. If a substantial number of candidates agree to participate in the trial, the list of the participating candidates may be provided to the client 36. If there is very few or no candidates who agree to participate in the trial, the client 36 is notified as shown in FIG. 1. Obviously, if the candidate contact process is performed by the client side as mentioned above, the online community may not be involved in the candidate contact process.

As described above, the invention provides an effective method of recruiting participants for clinical trials from a large pool of potential candidates, which results in significantly quicker drug trials.

While the invention has been described in terms of exemplary embodiments, those skilled in the art will recognize that the invention can be practiced with modifications in the spirit and scope of the appended claims. These examples given above are merely illustrative and are not meant to be an exhaustive list of all possible designs, embodiments, applications or modifications of the invention.

What is claimed is:

1. A method of recruiting clinical trial candidates using an online community, comprising the steps of:
creating an illness-specific online community;
creating membership accounts for members joining the online community;
receiving personal and medical information from the members;
categorizing and storing the received personal and medical information to a member database;
receiving information on a clinical trial from a client; and
searching the member database to determine candidates for the clinical trial.

2. The method of claim 1, further comprising a step of contacting the candidates to offer an opportunity to participate in the clinical trial.

3. The method of claim 1, further comprising a step of providing a list of the candidates who agree to participate in the clinical trial to the client.

4. The method of claim 1, further comprising a step of associating the personal and medical information to the corresponding membership accounts.

5. The method of claim 1, further comprising a step of creating a client account for the client.

6. The method of claim 5, further comprising a step of associating information on the clinical trial to the client account.

7. The method of claim 6, further comprising a step of categorizing and storing the information on the clinical trial to a client database.

8. The method of claim 1, wherein the personal information is at least one of the member's name, address, date of birth, gender, race, email address and photo.

9. The method of claim 1, wherein the medical information is at least one of the member's medical history, condition of interest, treatment of interest, medications, side effects, participation in past clinical trials, and interest in future clinical trials.

10. The method of claim 1, wherein the medical information comprises at least one of the member's prior clinical trial experiences and the member's interest in learning about clinical trials relevant to the member's condition.

11. The method of claim 1, further comprising a step of receiving sharing preference information from the client.

12. A method of recruiting clinical trial participants online, comprising the steps of:
creating a website dedicated to at least one specific illness, the website comprising a plurality of web pages linked to each other, the web pages including a home page, membership application page and member profile page;
displaying the membership application page containing an online membership application form when an online visitor indicates that he or she wants to be a member;
creating a membership account for the member responsive to a visitor's input;
collecting personal and medical information from the member;
associating the member's personal and medical information to the member's membership account;
categorizing and storing the member's personal and medical information to a member database;
receiving clinical trial information from a client;
comparing the clinical trial information to the member's personal and medical information to determine if the member is a candidate for the client's clinical trial; and
contacting the member to offer an opportunity to participate in the client's clinical trial.

13. The method of claim 12, further comprising a step of displaying the member profile page if the member indicates that he or she wants to add, delete or edit the personal and medical information.

14. The method of claim 12, wherein the web pages further include an information sharing preference page.

15. The method of claim 14, further comprising the steps of:
creating a client account for the client;
receiving client information from the client;
associating the client information and clinical trial information to the client account; and
categorizing and storing the client and clinical trial information to a client database.

16. The method of claim 14, wherein the personal information is at least one of the member's name, address, date of birth, gender, race, email address and photo.

17. The method of claim 14, wherein the medical information is at least one of the member's medical history, condition of interest, treatment of interest, medications, side effects.

18. The method of claim 12, wherein the medical information comprises at least one of the member's prior clinical trial experiences and the member's interest in learning about clinical trials relevant to the member's condition.
